(19) United States
(12) Patent Application Publication
(10) Pub. No.: US 2022/0249003 A1
(43) Pub. Date: Aug. 11, 2022

(54) ELECTROCARDIOGRAM MONITORING DEVICE FOR PETS

(71) Applicant: UJURA COMPANY Co, Ltd., Daegu (KR)

(72) Inventors: Yong Ju SHIM, Incheon (KR); Jong Woo PARK, Seoul (KR)

(73) Assignee: UJURA COMPANY Co, Ltd., Daegu (KR)

(21) Appl. No.: 17/533,370

(22) Filed: Nov. 23, 2021

(30) Foreign Application Priority Data
Feb. 9, 2021 (KR) 10-2021-0018112

(51) Int. Cl. A61B 5/282; A61B 5/321

(52) U.S. Cl. CPC A61B 5/282; A61B 5/1114; A61B 5/321

(57) ABSTRACT

Proposed is an electrocardiogram monitoring device for pets such as dogs and cats, the device including a pad that a pet climbs on a plurality of electrode bodies that are dispersedly disposed on an upper surface of the pad, and of which top portions are exposed, an electrode differentiation unit configured to select electrode bodies that are in contact with paw pads of the pet and that will receive electrical signals for electrocardiogram, and an electrocardiogram measurement unit configured to receive the electrical signals from the electrode bodies selected by the electrode differentiation unit and perform the electrocardiogram.

### CROSS REFERENCE TO RELATED APPLICATION

The present application claims priority to Korean Patent Application No. 10-2021-0018112, filed Feb. 9, 2021, the entire contents of which is incorporated herein for all purposes by this reference.

### BACKGROUND OF THE INVENTION

### Field of the Invention

The present disclosure relates to an electrocardiogram monitoring device for pets such as dogs and cats.

### Description of the Related Art

As the number of companion animals increases and awareness of animal welfare improves, the veterinary service market is growing significantly. Medical prevention activities applied to humans are gradually being applied to companion animals, and due to aging of companion animals, various diseases are arising and a wide range of treatment services are also developing accordingly.

An electrocardiogram (ECG) is an important diagnostic tool comparable to blood pressure measurement in diagnosing heart-related diseases. One of the methods for performing ECG testing for a companion animal in the conventional art is to attach a measuring probe to the body of the companion animal and do the ECG monitoring for a certain period of time. However, in the case of companion animals, it is virtually impossible to perform ECG properly because the fact that electrodes are attached to their body makes them struggle to drop them, and this causes a lot of stress to the animals. Alternatively, an ECG can be carried out by inducing a forced rest state by anesthesia, but it is often avoided for reasons such as the poor health of the companion animal or the cost burden due to the increased treatment time.

As another conventional technique, there is a method of observing the heart rate using a wearable device worn by a companion animal. It is a method that primarily uses a pair of electrodes or sensors that contact the neck and shoulder areas, yet this is just a simple heart rate measurement and not a means to accurately measure the heart condition.

### DOCUMENTS OF RELATED ART

(Patent Document 1) Korean Patent Application Publication No. 10-2019-0103626 (Sep. 5, 2019)

(Patent Document 2) Korean Patent Application Publication No. 10-2017-0101358 (Sep. 6, 2017)

(Patent Document 3) Korean Patent Application Publication No. 10-2009-0008778 (Jan. 22, 2009)

(Patent Document 4) Korean Patent Registration No. 10-2018887 (Aug. 30, 2019)

(Patent Document 5) Korean Patent Registration No. 10-1969050 (Apr. 9, 2019)

(Patent Document 6) Korean Patent Registration No. 10-1650779 (Aug. 18, 2016)

(Academic journal) “Smart-surface: Large scale textile pressure sensors arrays for activity recognition” (author Jingyuan Cheng et al., 2016)

### SUMMARY OF THE INVENTION

Accordingly, the present disclosure has been made keeping in mind the above problems occurring in the related art, and the present disclosure is intended to provide an electrocardiogram monitoring device for pets that can perform an electrocardiogram (ECG) without stressing a companion animal.

Other detailed objectives of the present disclosure will be clearly grasped and understood by experts or researchers in the art from the following description.

In order to achieve the above objective, according to an embodiment of the present disclosure, there is provided an electrocardiogram monitoring device for pets, the device including: a pad that a pet climbs on; a plurality of electrode bodies that are dispersedly disposed on an upper surface of the pad, and of which top ends are exposed; an electrode differentiation unit configured to select electrode bodies that are in contact with paw pads of the pet and that will receive electrical signals for electrocardiogram; and an electrocardiogram measurement unit configured to receive the electrical signals from the electrode bodies selected by the electrode differentiation unit and perform the electrocardiogram.

The electrode differentiation unit may include: a weight measuring means configured to measure weight distribution of the pet on the pad; a posture recognition module configured to recognize a paw shape from the weight distribution; and a selection module configured to select an electrode body that will receive an electrical signal for each foot when it is determined that the pet is in a prone position.

According to another embodiment, the electrode differentiation unit may include: a posture recognition module configured to identify the electrode body in contact with paw pads of each paw of the pet and determine an area where the paw exists; and a selection module configured to select the electrode body to receive the electrical signal for each area.

According to yet another embodiment, the electrode differentiation unit may include: a camera device configured to locate on an upper part of the pad and acquire an image of the pet on the pad; an image analysis module configured to analyze the image to estimate the position of paw pads of each paw of the pet; and a selection module configured to select an electrode body that will receive an electrical signal in an area corresponding to the estimated position of paw pads of each paw, when it is determined that the pet is in a prone position.

According to the above-described embodiments, the top ends of the electrode bodies may be protruding out of the pad and the electrode bodies may be configured to be elastically supported and able to move up and down.

The electrocardiogram monitoring device for pets may further include a heating unit configured to raise temperature of the upper surface of the pad.

According to the embodiment of the present disclosure, it is possible to perform an electrocardiogram without stressing a pet, thereby improving the reliability of the measurement result and improving the accuracy of diagnosis.

In addition, just by installing the device in a breeding facility, the electrocardiogram measurement is performed frequently, increasing the possibility of early detection and early treatment of heart-related diseases, thereby improving the breeding environment for pets.

Effects obtainable from the present disclosure may be non-limited by the above mentioned effects. Also, other unmentioned effects can be clearly grasped and understood by experts or researchers in the art from the following description or during the implementation of the present disclosure.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1 is a perspective view schematically showing an electrocardiogram monitoring device for pets according to the first embodiment of the present disclosure;

FIG. 2 is a perspective view separately showing the main part of the embodiment shown in FIG. 1;

FIG. 3 is a cross-sectional view schematically showing the main part of the embodiment shown in FIG. 1;

FIG. 4 is a cross-sectional view showing the main part of an electrocardiogram monitoring device for pets according to the second embodiment of the present disclosure;

FIG. 5 is a view schematically showing the weight distribution of a companion animal;

FIG. 6 is a view schematically showing the process of selecting an electrode body;

FIG. 7 is a view schematically showing a subject for measuring an electrocardiogram;

FIG. 8 is a perspective view showing an electrocardiogram monitoring device for pets according to the third embodiment of the present disclosure;

FIG. 9 is a cross-sectional view schematically showing the main part of an electrocardiogram monitoring device for pets according to the fourth embodiment of the present disclosure;

FIG. 10 is a view related to the operation of an electrode differentiation unit;

FIG. 11 is a view related to another operation of the electrode differentiation unit.

### DETAILED DESCRIPTION OF THE INVENTION

Hereinbelow, configuration, function, operation of an electrocardiogram monitoring device for pets according to the present disclosure will be described in detail with reference to the accompanying drawings. However, throughout the drawings and embodiments, reference numerals for the same or similar components will be used uniformly.

In addition, in the following description, the terms “first”, “second”, etc. are used to distinguish components having the same technical meaning for convenience. That is, any one configuration may be arbitrarily called a “first configuration” or a “second configuration”.

The accompanying drawings show embodiments according to the present disclosure, and the technical spirit of the present disclosure should not be construed as being limited through the accompanying drawings. If it can be interpreted that some or all of the figures shown in the drawings are not necessarily in the form, shape, and order required for the implementation of the disclosure from the point of view of an expert in this technical field, this does not limit the disclosure described in the claims.

A pet to which the electrocardiogram monitoring device according to an embodiment of the present disclosure is applied is an animal with exposed skin (paw pad) on the soles of its feet that is not covered with hair. It is especially applicable to dogs and cats with the behavioral trait of resting in a sitting or prone position with the soles of their feet on the floor or rest in a prone state.

Also, the terms “paw pad” and “foot pad”, which refer to the bare skin exposed on the soles of an animal, are used interchangeably.

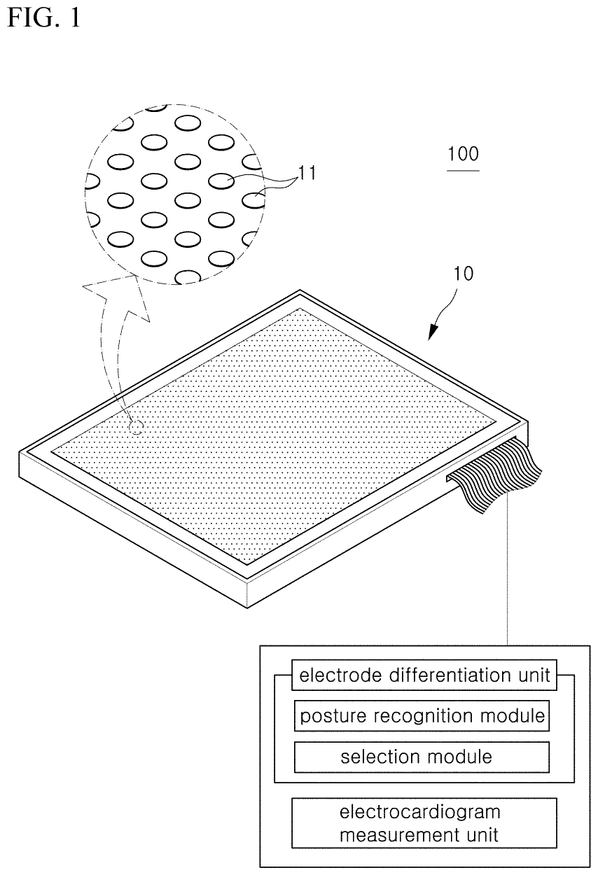

FIGS. 1 to 3 relate to an electrocardiogram monitoring device for pets according to the first embodiment of the present disclosure.

The electrocardiogram monitoring device for pets 100 according to the first embodiment includes a pad 100, an electrode body 11, an electrode differentiation unit, and an electrocardiogram measurement unit.

The upper surface of the pad 10 is a largely flat surface, on which a pet is placed. It is formed with a sufficient area according to the size of the pet so that the pet can voluntarily climb up and sit or lie on their belly.

A plurality of electrode bodies 11 are dispersedly disposed on the upper surface of the pad 10, and the top ends 111 of the electrode bodies are exposed on the upper surface of the pad 10. The electrode body 11 is a metal of a conductive material and is used as an electrode to receive electrical signals for electrocardiogram measurement by contacting the pet's skin.

In the drawings, the electrode bodies are arranged in a grid shape, but in other embodiments not shown, the dense arrangement structure of the electrode bodies may be variously changed.

The electrode bodies 11 are mounted on a circuit board 20, and through holes 31 corresponding to the electrode bodies are formed in a pad housing 30, so that the electrode bodies 11 protrude out of the pad and are exposed. The circuit board 20 is electrically connected to the electrode differentiation unit and the electrocardiogram measurement unit.

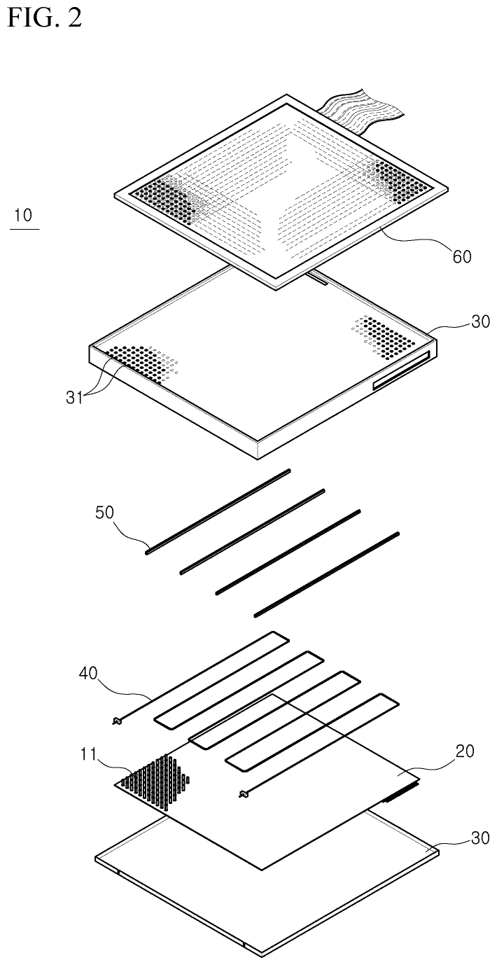

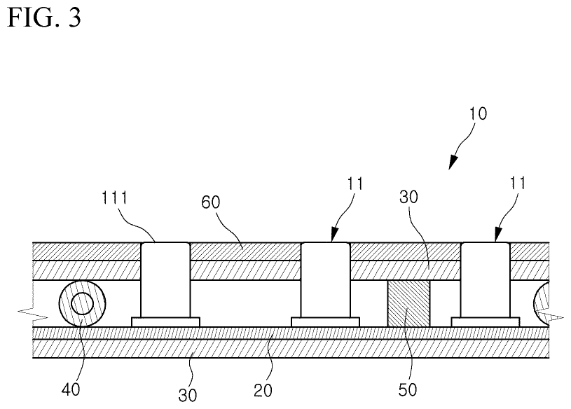

In FIGS. 2 and 3, the main components of the electrocardiogram monitoring device for pets 100 according to the first embodiment are shown.

The circuit board 20 on which the plurality of electrode bodies 11 are mounted is mounted on the pad housing 30 assembled up and down. A heating unit 40 and a support block 50 are mounted on the circuit board 20.

On the upper pad housing 30 in which the plurality of through holes 31 through which the electrode bodies 11 pass are formed, a weight measuring means for measuring weight distribution of the pet on the pad 10 is provided.

The weight measuring means 60 is a component of an electrode differentiation unit that selects electrode bodies for an electrocardiogram of a pet, and is largely in the form of a plate or a sheet.

A plurality of holes through which the electrode bodies pass are formed in the weight measuring means 60 corresponding to the through holes 31. The top ends 111 of the electrode bodies 11 pass through the through holes 31 of the pad housing 30 and the holes of the weight measuring means 60, and are exposed on the upper surface of the pad 10. Here, the top ends of the electrode bodies may have a height corresponding to the upper surface of the pad or may protrude slightly higher than that.

The heating unit 40 is operated to the extent of warming the upper surface temperature of the pad, and a belt-type carbon heating wire that generates heat according to power supply may be used. By taking advantage of the cat's preference for a warm place, you can induce the cat to climb on its own on a pad with a warm top and stay there for a long time. The heating unit 40 may be omitted depending on the behavioral characteristics of the pet to be measured.

The support block 50 is a structure that is disposed in the floating space of the pad housing to prevent the central portion of the pad housing from being pressed. This support block may be omitted depending on the design structure and the like of the pad housing.

The electrode differentiation unit selects electrode bodies 11 to receive the electric signals for the electrocardiogram measurement of the pet from among the electrode bodies provided in the pad 10.

In the first and second embodiments shown in FIGS. 1 to 4, the electrode differentiation unit includes the weight measuring means 60, a posture recognition module, and a selection module.

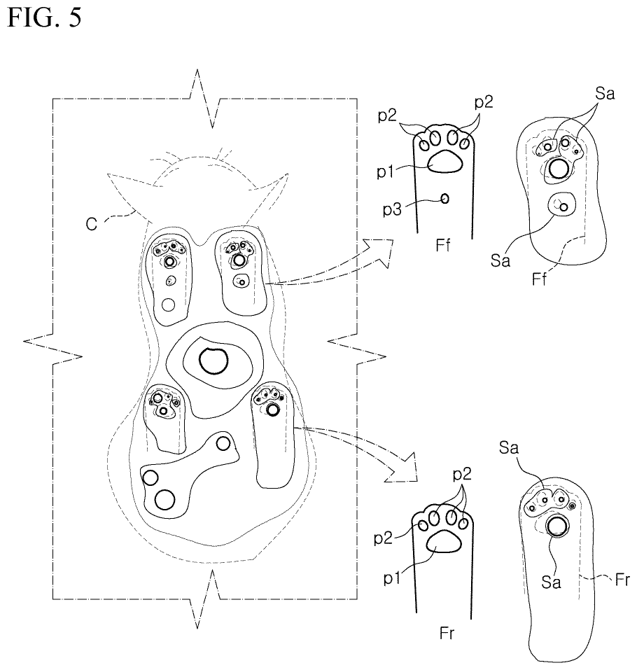

The weight measuring means 60 is to measure the weight distribution of the pet on it, and the weight distribution may be expressed as an area on a plane. Consequently, as shown in FIG. 5, the weight distribution may be expressed in the form of a contour line or may be expressed by color.

Such a weight measuring means may be implemented using a distributed load measuring device described in Korean Patent Application Publication No. 10-2009-0008778 or a sensor array introduced in “Smart-surface: Large scale textile pressure sensors arrays for activity recognition” (author Jingyuan Cheng et al.) published in an academic journal.

Alternatively, the weight measuring means may be implemented by installing a pressure sensor for each electrode body, and mapping the output value of each pressure sensor as the pet climbs on the pad to the position of the electrode bodies.

As described above, the weight measuring means may be configured using one of various techniques that can measure the weight distribution of the pet on the pad.

The posture recognition module is to determine the shape of the four paws from the weight distribution of the pet measured by the weight measuring means 60. Furthermore, the posture of the pet may be estimated.

Since the posture identification module aims to find the sole of the foot, it is assumed that the pet on the pad is in a posture with the sole of the foot on the pad. When the pet is standing on the pad, sitting with its feet on the pad, or lying face down, there is a high probability that its paws are in contact with the pad.

The posture identification module starts when a weight change is detected, performs the corresponding operation until it successfully determines the shape of the paw to be described below, and when the weight is removed, the operation stops as the pet has left the pad.

FIG. 5 shows an example in which the weight distribution generated as a cat C climbs on the weight measuring means is illustrated in the form of a contour line. The cat C on the pad is in a prone position with all four paws on the floor. This prone position is characteristic of cats and is generally referred to as the “catloaf” position. Cats have the characteristic of staying in this position for more than several minutes, which long enough to perform an electrocardiogram.

While in the prone position, there is a high probability that the soles of all four feet are in contact with the pads. At this time, the weight of the cat C is transmitted to the weight measuring means through the four soles and abdomen, and information about this weight distribution is transmitted to the posture identification module.

On each paw of the cat C, four digital pads p2 are located radially in front centering on a metacarpal pad p1. In particular, it is known that only the two forefeet have a carpal pad p3 symmetrically with the digital pads. These foot pads or paw pads are prominently protruding, and there is hair therebetween, so they are clearly distinguished.

The posture identification module detects the weight distribution information measured through the weight measuring means and the shape and position of the paw pads for each paw, and matches the detected information with the physiological information to determine the shape of the paw and its position.

Specifically, based on the data on the size difference, number, and arrangement relationship of the metacarpal pad p1 and the digital pads p2 revealed in animal physiology, the shape, position, and direction of the paw are calculated by matching the data with weight distribution. Here, to supplement data on physiological characteristics, individual characteristic information such as breed, age, and weight of each pet may be reflected.

In addition, by detecting whether there is a weight distribution according to the carpal pad p3, it is possible to determine whether the paw is the forepaw or the hindpaw.

If three paws are recognized, the position of the remaining paw can be estimated, and the shape of the remaining paw can be easily determined from the estimated position.

Further, it is possible to distinguish between the forepaws and the hindpaws, and infer the direction (head and tail position) that the cat C is facing from the relative distance information between them, and infer what the cat's current posture is.

Posture information is stored together with the electrocardiogram measurement result, so that it is possible to refer to whether the posture is the same when comparing with the electrocardiogram measured in the past.

Whether a cat is in a prone position may be recognized as a state suitable for performing electrocardiogram (a state in which the cat maintains the same position for a long period of time) from the fact that it stays in the prone position for a long period of time, and may be regarded as a resting state, which may be taken into account when veterinarians evaluate the reliability of the electrocardiogram.

On the other hand, the selection module selects electrode bodies to receive an electrical signal for electrocardiogram measurement for each paw.

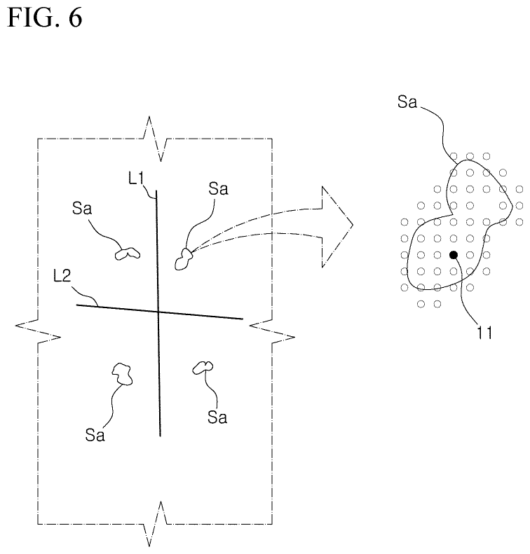

Referring to FIG. 6, the selection module draws two straight lines that divide the space where each paw is located through the position information of the paws, and an extraction area Sa in which the electrode body 11 and the paw pad contact is extracted for each space.

Here, the area itself recognized as a paw pad in the posture identification module may be selected as an extraction area in which the electrode body and the foot pad are in contact.

Alternatively, considering the area recognized as a paw pad and the weight distribution contour line together, the intersection area where the area to which a weight relatively higher than the surrounding area is exerted (bounded by the contour line) and the area recognized as a paw pad overlap may be selected as the extraction area.

When multiple extraction areas are derived from one space, only one with the largest size or the heaviest weight among them may be selected as the extraction area.

Thereafter, the selection module selects any one of the plurality of electrode bodies included in the extraction area Sa selected for each space or spanned by the boundary as the electrode body 11 for performing electrocardiogram.

After testing the electrode bodies corresponding to the extraction area in turn, you can select the one most suitable for electrocardiogram measurement. The testing may be performed by comparing and evaluating the strength of the electrical signal for each electrode body or confirming whether the intended graph is secured by the electrocardiogram measurement unit, which will be described later.

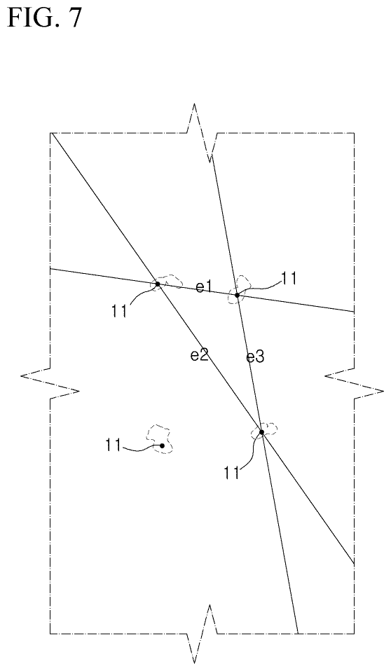

The electrocardiogram measurement unit receives electrical signals from the selected electrode bodies in each space and performs electrocardiogram measurement. Referring to FIG. 7, as the electrode body of the left forefoot, the electrode body of the right forefoot, the electrode body of the left hindfoot, and the electrode body of the right hindfoot are specified, a limb lead electrocardiogram measurement using the first lead, the second lead, and the third lead is performed. The measurement algorithm and signal processing configuration of this lead method may be the same as those known in the art.

The electrocardiogram measurement result may be stored on a server, etc., and presented to the breeder in the form of a graph.

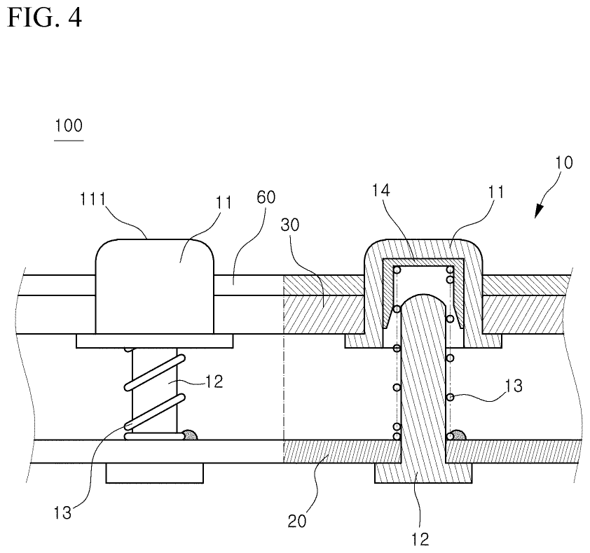

FIG. 4 relates to the electrocardiogram monitoring device according to the second embodiment of the present disclosure, and in particular relates to the configuration of the electrode body.

The electrocardiogram monitoring device 100 according to the second embodiment includes a pad 10, an electrode body 11, an electrode differentiation unit and an electrocardiogram measurement unit, thus including the technical features of the above-described embodiment within a range that does not conflict with the contents described below.

In the second embodiment, the electrode bodies 11 protrude from the top of the pad 10 and are elastically supported, so, as the pet pushes it, the electrode bodies 11 come into contact with the pet's skin while being elastically supported.

Specifically, a guide column 12 is erected on the circuit board 20, and a spring 13 made of a conductive material is mounted on its outer peripheral surface. The upper portion of the spring 13 is attached to a cap 14 bound to the inside of the electrode body 11 with a conductive adhesive. The lower portion of the spring 13 is electrically connected to a circuit pattern (not shown) of the circuit board 20 and solder or the like.

Accordingly, while it is possible to transmit an electrical signal through the electrode body 11 to the circuit board 20 through the spring 13, the electrode body 11 is elastically supported by the spring 13.

Although not shown, various techniques for elastically supporting the electrode body may be applied, such as forming a curved pattern on the lower portion of the electrode body to retain elasticity by the electrode body itself without the aforementioned spring.

Since the elastically supported electrode bodies 11 tend to protrude from the upper surface of the pad, they actively come into contact with the bare skin of the pet on the pad 10. As the contact with the skin is ensured, the operation reliability of the electrode bodies is improved.

The electrode bodies 11, which are elastically supported and protrude above the pad, may come into contact with the skin by penetrating the pet's hair. Therefore, compared to other embodiments that involve no elastic support, the electrode bodies can come into contact with the skin of the paw pad even at the edge of the paw pad, which is covered by hair. As a result, the probability of success in determining the shape of the paw increases as the paw pad is greatly recognized.

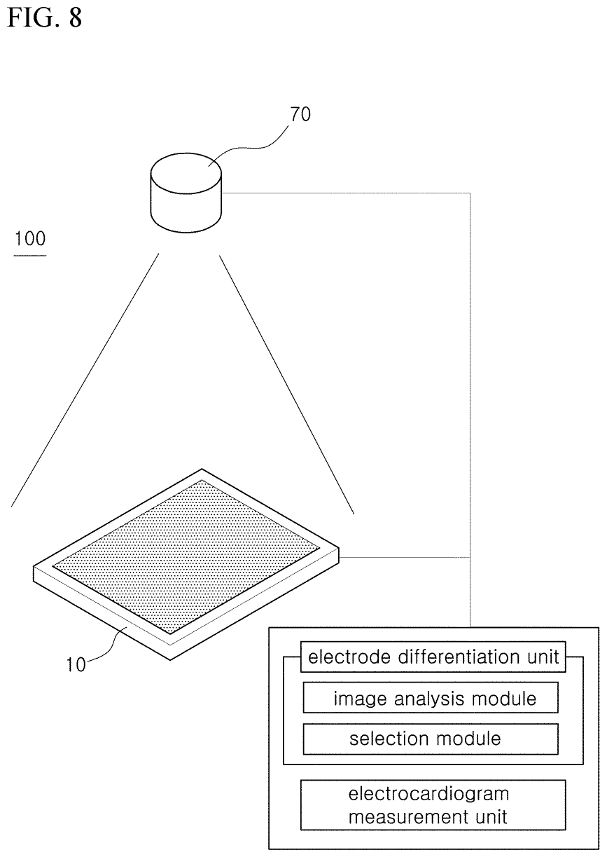

FIG. 8 relates to an electrocardiogram monitoring device according to the third embodiment of the present disclosure.

The electrocardiogram monitoring device according to the third embodiment includes a pad 10, an electrode body 11, an electrode differentiation unit and an electrocardiogram measurement unit, thus including the technical features of the above-described embodiment within a range that does not conflict with the contents described below.

The electrode differentiation unit according to the third embodiment includes a camera device 70, an image analysis module, and a selection module.

The camera device 70 is positioned high apart from the upper surface of the pad 10, and takes an image of a pet mounted on the pad 10. While in FIG. 8, one camera device 70 is shown as being positioned vertically above the pad, a plurality of camera devices may be installed with different viewing angles of the pad.

The image analysis module receives the image photographed by the camera device 70, performs image analysis, and estimates the position of the paw pad of each foot of the pet.

Specifically, the image analysis module recognizes the shape of the cat through edge extraction from the image, and estimates the position of the four paws and the paw pad on each paw through posture analysis. By matching the estimated position of the paw pad with the coordinate value of the pad, the area where the paw pad is expected to be located is calculated as the coordinate value.

To this end, the image analysis module utilizes known image analysis technologies such as image edge extraction, object recognition tools and body part detection using machine learning, self-estimation algorithms (Korean Patent Registration No. 10-2018887, Korean Patent Registration No. 10-1969050), and image analysis tools including variable posture (Korean Patent Registration No. 10-1650779).

The image analysis module provides the selection module with information on the area where the paw pad is estimated to exist.

The selection module, as shown in FIG. 6, selects an electrode body to receive an electrical signal for electrocardiogram measurement from among the electrode bodies distributed in the area corresponding to the estimated position of the paw pad of each foot. A specific selection method may be the same as in the above-described embodiment.

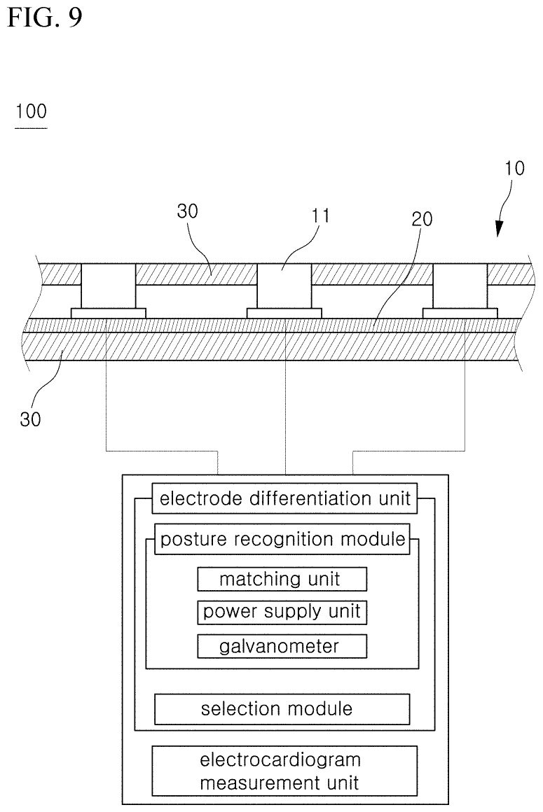

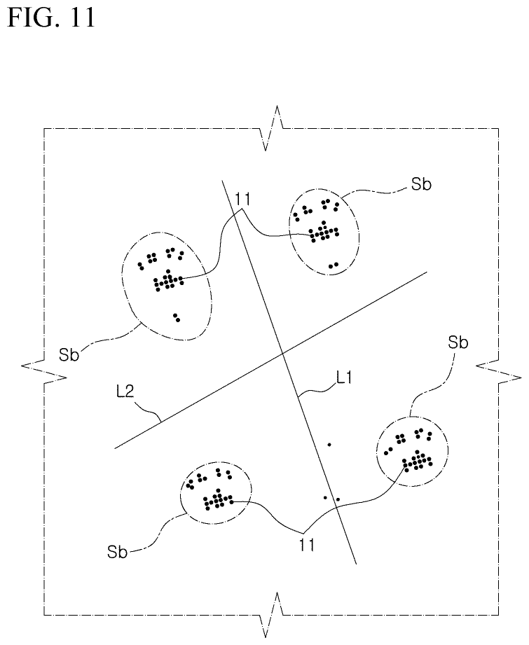

FIGS. 9 to 11 relate to an electrocardiogram monitoring device according to the fourth embodiment of the present disclosure.

The electrocardiogram monitoring device according to the fourth embodiment includes a pad 10, an electrode body 11, an electrode differentiation unit and an electrocardiogram measurement unit, thus including the technical features of the above-described embodiment within a range that does not conflict with the contents described below.

Referring to FIG. 9, a circuit board 20 on which a plurality of electrode bodies 11 are mounted is seated in the pad housing 30, and the top end of the electrode body 11 is exposed at a virtually equal height to the upper surface of the pad 10.

The electrode differentiation unit according to the fourth embodiment includes a posture identification module and a selection module.

The posture identification module detects the electrode bodies in contact with the paw pad among the electrode bodies provided in the pad 10, and determines the area where the paws exist therefrom.

Specifically, the process can be done by selecting two adjacent electrode bodies and mapping whether they are energized or not through an energization test between them. To this end, the posture identification module includes a matching unit that sequentially selects a pair of electrode bodies one by one, a power supply unit that applies a current to any one of the selected electrode bodies, and a galvanometer that checks whether the electrode bodies are energized.

Here, a pair of electrode bodies may be preset, or the matching unit may select one electrode body (for example, located at the top left) as a reference, then select two left and right or up and down to perform an energization test.

If energized, it is considered that the two electrode bodies are in contact with the paw pad, and if not energized, they are treated as not being in contact with the paw pad.

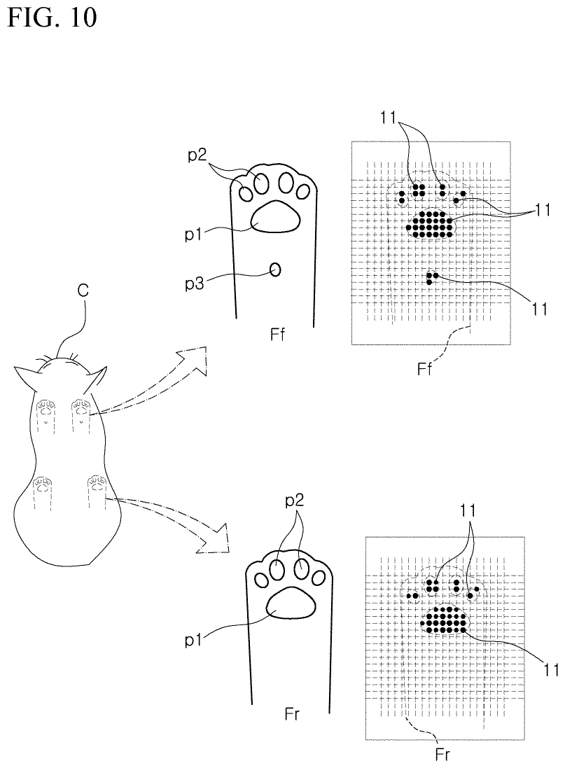

Referring to FIG. 10, in the cat's forefoot Ff, the electrode bodies 11 are energized and are detected in the metacarpal pad p1, digital pad p2, and carpal pad p3. Also, the electrode bodies 11 in contact with the metacarpal pad p1 and the digital pad p2 are detected in the hindfoot Fr. The paw is recognized by analyzing the pattern formed by the energized electrode bodies.

In addition, it is possible to distinguish the forefoot and the hindfoot from the electrode body 11 in contact with the carpal pad p3. The orientation of the pet's head and tail is also determined.

The area Sb where each paw is located can be specified from the pattern of electrode bodies following the arrangement structure of the paw pad. By examining whether the shape of the area of each paw is symmetrical or square, the task of setting the area corresponding to the four paws may be supplemented. The active electrode body other than the four areas Sb collected in this way is ignored in the subsequent process.

The selection module selects an electrode body to receive an electrical signal for electrocardiogram measurement for each area. Here, the criteria for selecting the electrode body are the same as in the above-described embodiment.

Thereafter, the electrocardiogram measurement unit receives an electric signal from the selected electrode body and performs an electrocardiogram measurement.

The electrocardiogram monitoring device for pets according to the present disclosure does not cause stress on the pet as it does not put any equipment on the pet or attach electrodes. Therefore, it is possible to make an ideal diagnosis by observing an electrocardiogram on the pet in a relaxed state. In addition, electrocardiogram measurement is possible even in situations in which the body of the test subject may not be restrained due to injuries, disabilities, aging, and the like.

By installing the device in a breeding facility, the electrocardiogram measurement can be performed frequently. Also, continuous monitoring is possible throughout the growth period, so the occurrence of heart-related diseases can be quickly identified.

What is claimed is:

1. An electrocardiogram monitoring device for pets, the device comprising:
a pad that a pet climbs on;
a plurality of electrode bodies that are dispersedly disposed on an upper surface of the pad, and of which top ends are exposed;
an electrode differentiation unit configured to select electrode bodies that are in contact with paw pads of the pet and that will receive electrical signals for electrocardiogram; and
an electrocardiogram measurement unit configured to receive the electrical signals from the electrode bodies selected by the electrode differentiation unit and perform the electrocardiogram.

2. The electrocardiogram monitoring device for pets of claim 1, wherein the electrode differentiation unit comprises:
a weight measuring means configured to measure weight distribution of the pet on the pad;
a posture recognition module configured to recognize a paw shape from the weight distribution; and
a selection module configured to select an electrode body that will receive an electrical signal for each foot when it is determined that the pet is in a prone position.

3. The electrocardiogram monitoring device for pets of claim 1, wherein the electrode differentiation unit comprises:
a posture recognition module configured to identify the electrode body in contact with paw pads of each paw of the pet and determine an area where the paw exists; and
a selection module configured to select the electrode body to receive the electrical signal for each area.

4. The electrocardiogram monitoring device for pets of claim 1, wherein the electrode differentiation unit comprises:
a camera device configured to locate on an upper part of the pad and acquire an image of the pet on the pad;
an image analysis module configured to analyze the image to estimate the position of paw pads of each paw of the pet; and
a selection module configured to select an electrode body that will receive an electrical signal in an area corresponding to the estimated position of paw pads of each paw, when it is determined that the pet is in a prone position.

5. The electrocardiogram monitoring device for pets of claim 1, wherein the top ends of the electrode bodies are protruding out of the pad and the electrode bodies are elastically supported and able to move up and down.

6. The electrocardiogram monitoring device for pets of claim 1, further comprising:
a heating unit configured to raise temperature of the upper surface of the pad.
